(19) United States
(12) Patent Application Publication
(10) Pub. No.: US 2009/0239960 A1
(43) Pub. Date: Sep. 24, 2009

(54) METHODS AND SYSTEMS FOR FISCHER TROPSCH REACTOR LOW PRODUCT VARIATION

(76) Inventors: Paul Steven Wallace, Katy, TX (US); Arnaldo Frydman, Houston, TX (US)

Correspondence Address: JOHN S. BEULICK (17851);ARMSTRONG TEASDALE LLP, ONE METROPOLITAN SQUARE, SUITE 2600, ST. LOUIS, MO, 63102-2740, US

(21) Appl. No.: 12/054,208

(22) Filed: Mar. 24, 2008

(51) Int. Cl. C07C 27/00

(52) U.S. Cl. 518/700

(57) ABSTRACT

Methods and systems for operating a carbon to liquids system are provided. The method includes receiving a flow of syngas, shifting the syngas to increase an H₂/CO ratio of the syngas, mixing hydrogen with the shifted syngas to increase the H₂/CO ratio, reacting the hydrogen/shifted syngas mixture with a catalyst in a vessel at a pressure of approximately 600 psia such that approximately 40% of the hydrogen/shifted syngas mixture is converted, recycling an un-reacted hydrogen/shifted syngas mixture to the vessel, and recycling naphta to a catalyst recovery system

### BACKGROUND OF THE INVENTION

This invention relates generally to carbon to liquids systems, and more specifically to methods and systems for minimizing liquid product variation from a Fischer-Tropsch reactor portion of the system.

The terms C5+ and “liquid hydrocarbons” are used synonymously and refer to hydrocarbons or oxygenated compounds having five (5) or greater numbers of carbons, including for example pentane, hexane, heptane, pentanol, pentene, and which are liquid at normal atmospheric conditions.

The terms C4− and “gaseous hydrocarbons” are used synonymously and refer to hydrocarbons or oxygenated compounds having four (4) or fewer numbers of carbons, including for example methane, ethane, propane, butane, butanol, butene, propene, and which are gaseous at normal atmospheric conditions.

Modern Fischer-Tropsch (FT) units have been optimized for synthesis gas (syngas) production from natural gas, also known as Gas-to-Liquids process (GTL). Generally, syngas refers to a mixture of mainly H₂ and CO, plus some CO₂, all at various proportions. The FT reactor is operated at relatively high residence times, high per pass conversion, and H₂/CO ratios below the consumption ratio to improve C5+ selectivity and minimize C4− selectivity, i.e. natural gas and liquefied petroleum gas (LPG) production. The remote location of most carbon to liquids plants makes natural gas and (LPG) co-production economically unattractive, due to high transportation costs.

Minimizing natural gas and LPG production result in a significant fraction (30-40%) of the FT liquids being converted to wax. This wax must then be converted back to diesel range products (generally C10-C20 range) using a separate hydrocracking reactor. Also, the high per pass conversion that is used to maximize C5+ production limits the pressure of the FT reactor and byproduct water partial pressure increases with conversion and total pressure. Such high water partial pressure may cause deactivation of the catalyst by oxidizing the active catalyst sites, while low water partial pressure may cause competitive adsorption among water, CO and H₂ molecules on the catalyst active site thus reducing (CO+H₂) conversion. Iron-based FT catalysts, in particular, can be greatly affected by water, while cobalt-based FT catalysts tend to be more resistant to oxidation by water Generally, the water volume % in the reaction media should be under the range of 15-25%, beyond which the catalyst deactivation effect is known to be quite extensive.

Other carbonaceous fuels may also be used to provide the syngas input to the FT process. However, undesirable product variation is caused by the characteristics of modern FT gas to liquids systems described above.

### BRIEF DESCRIPTION OF THE INVENTION

In one embodiment, a method for operating a carbon to liquids system includes receiving a flow of syngas, shifting the syngas to increase an H₂/CO ratio of the syngas, mixing hydrogen with the shifted syngas to increase the H₂/CO ratio, reacting the hydrogen/shifted syngas mixture with a catalyst in a vessel at a pressure of approximately 600 psia such that approximately 40% of the hydrogen/shifted syngas mixture is converted, and recycling an un-reacted hydrogen/shifted syngas mixture to the vessel.

In another embodiment, a carbon to liquids system includes a source of syngas, a vessel configured to shift the syngas to increase an H₂/CO ratio of the syngas, the vessel coupled in flow communication downstream of the source of syngas, a source of gas including hydrogen coupled in flow communication with the shifted syngas, the source of gas configured to be mixed with the shifted syngas to increase the H₂/CO ratio of the shifted syngas, a vessel including an inlet and an outlet, the inlet configured to receive the gas and shifted syngas mixture, the vessel including a catalyst configured to facilitate a Fischer-Tropsch synthesis reaction at a pressure of approximately 600 psia such that approximately 40% of the hydrogen/shifted syngas mixture is converted, and a recycle path communicatively coupled between the outlet and inlet configured to channel an un-reacted hydrogen/shifted syngas mixture to the vessel inlet.

In yet another embodiment, a system for generating liquid hydrocarbons from gaseous reactants includes a source of syngas including hydrogen and carbon monoxide in a ratio of between approximately 1.4 and approximately 1.8, a shift reactor configured to shift the syngas to increase an H₂/CO ratio of the syngas, the vessel coupled in flow communication downstream of the source of syngas, a source of gas including hydrogen coupled in flow communication with the shifted syngas, the source of gas configured to be mixed with the shifted syngas to increase the H₂/CO ratio of the shifted syngas to between approximately 1.9 to approximately 2.3, a vessel including an inlet and an outlet, the inlet configured to receive the gas and shifted syngas mixture, the vessel including a catalyst configured to facilitate a Fischer-Tropsch synthesis reaction at a pressure of approximately 600 psia such that approximately 40% of the hydrogen/shifted syngas mixture is converted, and a recycle path communicatively coupled between the outlet and inlet, the recycle path configured to channel an un-reacted hydrogen/shifted syngas mixture to the vessel inlet.

### BRIEF DESCRIPTION OF THE DRAWINGS

FIG. 1 is a schematic diagram of an exemplary known integrated gasification combined-cycle (IGCC) power generation system; and

FIG. 2 is a schematic diagram of a portion of an exemplary coal to liquids processing system in accordance with an embodiment of the present invention.

### DETAILED DESCRIPTION OF THE INVENTION

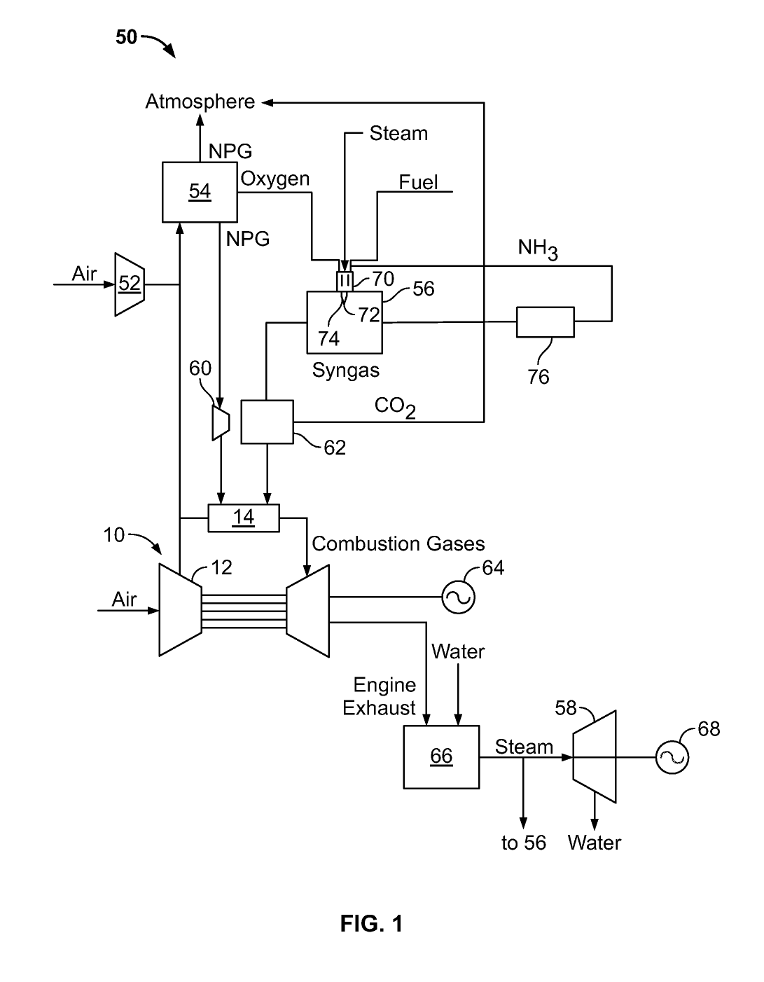

FIG. 1 is a schematic diagram of an exemplary known integrated gasification combined-cycle (IGCC) power generation system 50. IGCC system 50 generally includes a main air compressor 52, an air separation unit 54 coupled in flow communication to compressor 52, a gasifier 56 coupled in flow communication to air separation unit 54, a gas turbine engine 10, coupled in flow communication to gasifier 56, and a steam turbine 58. In operation, compressor 52 compresses ambient air. The compressed air is channeled to air separation unit 54. In some embodiments, in addition or alternative to compressor 52, compressed air from gas turbine engine compressor 12 is supplied to air separation unit 54. Air separation unit 54 uses the compressed air to generate oxygen for use by gasifier 56. More specifically, air separation unit 54 separates the compressed air into separate flows of oxygen and a gas by-product, sometimes referred to as a “process gas”. The process gas generated by air separation unit 54 includes nitrogen and will be referred to herein as “nitrogen process gas” (NPG). The nitrogen process gas may also include other gases such as, but not limited to, oxygen and/or argon. For example, in some embodiments, the nitrogen process gas includes between about 95% and about 100% nitrogen. The oxygen flow is channeled to gasifier 56 for use in generating partially combusted gases, referred to herein as “syngas” for use by gas turbine engine 10 as fuel, as described below in more detail. In some known IGCC systems 50, at least some of the nitrogen process gas flow, a by-product of air separation unit 54, is vented to the atmosphere. Moreover, in some known IGCC systems 50, some of the nitrogen process gas flow is injected into a combustion zone (not shown) within gas turbine engine combustor 14 to facilitate controlling emissions of engine 10, and more specifically to facilitate reducing the combustion temperature and reducing nitrous oxide emissions from engine 10. IGCC system 50 may include a compressor 60 for compressing the nitrogen process gas flow before being injected into the combustion zone.

Gasifier 56 converts a mixture of fuel, a carbonaceous substance, the oxygen supplied by air separation unit 54, steam, and/or limestone into an output of syngas for use by gas turbine engine 10 as fuel. Although gasifier 56 may use any fuel, in some known IGCC systems 50, gasifier 56 uses coal, petroleum coke, residual oil, oil emulsions, tar sands, and/or other similar fuels. In some known IGCC systems 50, the syngas generated by gasifier 56 includes carbon dioxide. The syngas generated by gasifier 56 may be cleaned in a clean-up device 62 before being channeled to gas turbine engine combustor 14 for combustion thereof or may be channeled to other systems for further processing, for example, to a Fischer-Tropsch synthesis reaction system for conversion to liquid hydrocarbons. Alternatively, a portion of the cleaned syngas after clean-up device 62, may be channeled to gas turbine engine combustor 14, while another portion may be channeled to a Fischer-Tropsch synthesis reaction system for conversion to liquid hydrocarbons. The portion ratio is dependant on the particular application. Carbon dioxide may be separated from the syngas during clean-up and, in some known IGCC systems 50, vented to the atmosphere, recycled to injection nozzle 70 of gasifier 56 (not shown), compressed and sequestered for geological storage (not shown), and/or processed to industrial use gases (not shown). The power output from gas turbine engine 10 drives a generator 64 that supplies electrical power to a power grid (not shown). Exhaust gas from gas turbine engine 10 is supplied to a heat recovery steam generator 66 that generates steam for driving steam turbine 58. Power generated by steam turbine 58 drives an electrical generator 68 that provides electrical power to the power grid. In some known IGCC systems 50, steam from heat recovery steam generator 66 is supplied to gasifier 56 for generating the syngas.

In the exemplary embodiment, gasifier 56 includes an injection nozzle 70 extending through gasifier 56. Injection nozzle 70 includes a nozzle tip 72 at a distal end 74 of injection nozzle 70. Injection nozzle 70 further includes a port (not shown in FIG. 1) that is configured to direct a stream of fluid proximate nozzle tip 72 such that the stream of fluid facilitates reducing a temperature of at least a portion of nozzle tip 72. In the exemplary embodiment, injection nozzle 70 is configured to direct a stream of ammonia proximate nozzle tip 72 such that the stream of ammonia facilitates reducing a temperature of at least a portion of nozzle tip 72.

In the exemplary embodiment, IGCC system 50 includes a syngas condensate stripper configured to receive condensate from a stream of syngas discharged from gasifier 56. The condensate typically includes a quantity of ammonia dissolved in the condensate. At least a portion of the dissolved ammonia is formed in gasifier 56 from a combination nitrogen gas and hydrogen in gasifier 56. To remove the dissolved ammonia from the condensate the condensate is raised to a temperature sufficient to induce boiling in the condensate. The stripped ammonia is discharged from stripper 76 and returned to gasifier 56 at a pressure higher than that of the gasifier, to be decomposed in the relatively high temperature region of the gasifier proximate nozzle tip 72.

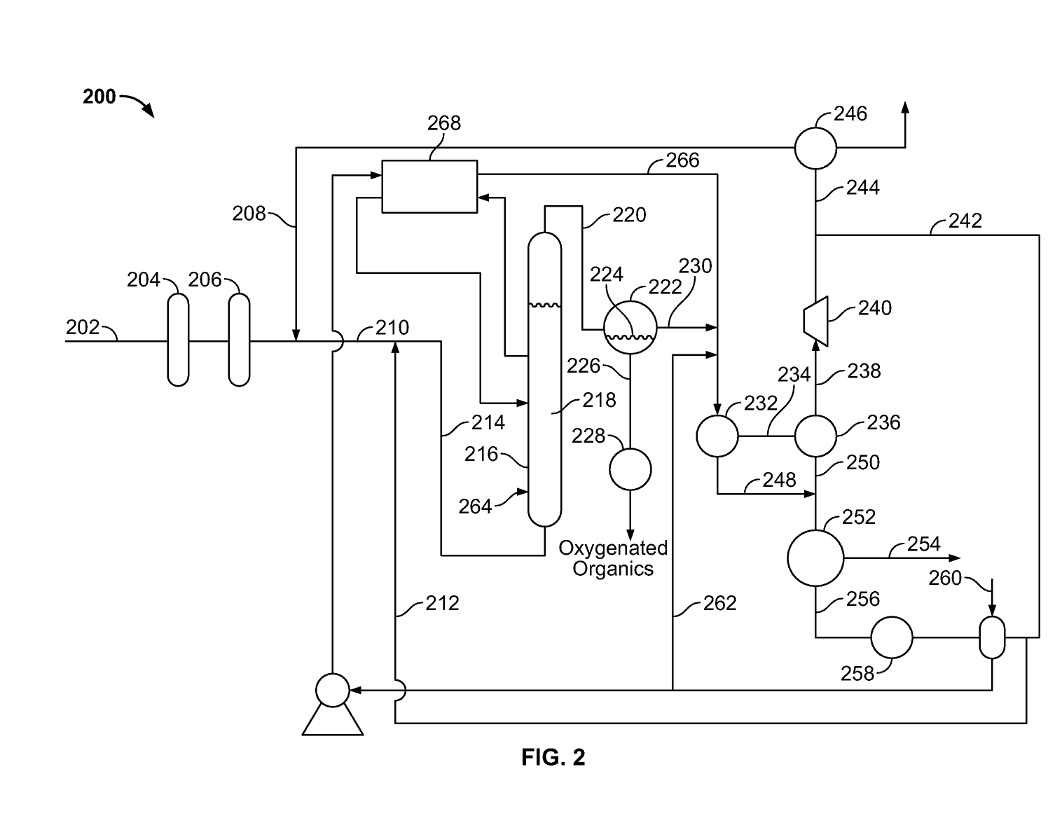

FIG. 2 is a schematic diagram of a portion of an exemplary coal to liquids processing system 200 in accordance with an embodiment of the present invention. Known commercial GTL reactors are generally operated at H₂/CO ratios of approximately 1.6 to optimize the C5+ selectivity. The kinetics of the FT reaction is improved at higher H₂/CO ratios. In the exemplary embodiment, using an H₂/CO ratio of 2.1 (FT consumption ratio), approximately one third less catalyst and reaction volume are used compared to an H₂/CO ratio of 1.6.

A flow of syngas 202 from a gasification process such as but, not limited to a coal gasification process, is prepared to an H₂/CO ratio of approximately 1.85 by shifting at least a portion of the syngas in a shift reactor 204 and removing essentially all of the CO₂, H₂S, and COS using, for example, a solvent and absorbent based system 206. The CO₂, H₂S, and COS removed streams in system 206 are not shown. Recycle hydrogen from a flow of tail gas 208 increases an H₂/CO ratio of a flow of feed gas 210 to approximately 2.10.

The flow of feed gas 210 is mixed with a flow of recycle gas 212 and a flow of mixed feed gas 214 is channeled to the bottom of a Fischer-Tropsch synthesis reactor 216. In the exemplary embodiment, Fischer-Tropsch (FT) synthesis reactor 216 comprises a slurry bubble column reactor (SBCR) type. Approximately 40% of the CO and hydrogen are converted into FT distillates and water in vapor form and FT wax in liquid form in SBCR 216.

The Fischer-Tropsch reaction for converting syngas, which is composed primarily of carbon monoxide (CO) and hydrogen gas (H₂), is characterized by the two following general reactions, which produce paraffinic hydrocarbons (reaction 1) and olefinic hydrocarbons (reaction 2):

(2n+1)H₂+nCO→CₙH₂ₙ₊₂+nH₂O (1)

2nH₂+nCO→CₙH₂ₙ+nH₂O (2)

Mixed feed gas 214 is fed to the bottom of SBCR 216 and distributed into a slurry 218 comprising liquid wax and catalyst particles. As the gas bubbles upwards through slurry 218, it is diffused and converted into more wax by the FT reaction, which is exothermic. The heat generated by the FT reaction is removed through cooling coils (not shown) where steam is generated for use elsewhere in system 200. SBCR 216 operates at a relatively high pressure of approximately 600 psia, but with a low per pass conversion of approximately 40% such that the water partial pressure is low enough to substantially reduce oxidizing and deactivating the catalyst. Generally, the water partial pressure in the mixture should be such that volume % content is less than 15-25%, depending on catalyst type.

A flow of FT distillates and water vapor 220 are condensed in a pump around condenser 222. A chimney tray 224 positioned internally to condenser 222 collects the condensed water and FT distillate and includes baffles (not shown) to provide gravity separation of the FT distillate from the water phases.

A flow of hot condensed water 226 from the baffles is channeled to a stripper 228 for separation of oxygenates and other organics. A flow of hot FT distillate 230 is channeled to a distillate stripper 232 for removal of dissolved gases, water, and lighter hydrocarbons. A flow of the dissolved gases, water, and lighter hydrocarbons 234 is condensed in a condenser 236 and a flow of remaining vapor 238 is compressed in a compressor 240 and combined with a FT tail gas stream 242 and a flow of the mixture 244 is channeled to a gas separation system 246. Recycled rich-hydrogen stream 208 from gas separation system 246 is channeled to feed gas 210.

A flow of stripped distillate 248 and a flow of hydrocarbon condensate 250 are channeled to an atmospheric distillation column 252 for fractionation to a flow of finished products 254 in an atmospheric distillation column.

A flow of condensed water 256 is routed to a stripper 258. A flow of syngas and hydrocarbon vapor from a water condensing section (not shown) of stripper 258 is contacted with a cooled high boiling product stream (lean oil) 260. Lean oil 260 absorbs any remaining light naphtha fraction in the FT reactor product gas and cools the gas stream. A flow of naphtha 262 is then routed to the FT distillate stripper 232 and atmospheric distillation column 252 for lean oil recovery and recycle. A majority of gas 212 from the lean oil absorber section is recycled back to the syngas feed line 210 and the remainder is removed as tail gas 242.

Alternatively, all or a portion of the naphta stream 262 is pumped to near 600 psia and routed to catalyst recovery system 268. Under these conditions, naphta (C5-C8 paraffinic hydrocarbons) becomes a supercritical solvent and will enhance solubility and removal of heavy waxes filling the pores of the catalyst particles, thus providing more efficient catalyst recovery. This will enable higher probability of chain growth since it will promote the reappearance of vacant sites on the catalyst surface, thereby providing accessibility for the re-adsorption of olefins and subsequent chain growth towards the desired C5+ selectivity range.

Accordingly, in operation a bubble column reaction section 264 of Fischer-Tropsch synthesis reactor 216 includes a reduced liquid height requirement and pump around condenser 222 has a relatively low pressure drop permitting a relatively high recycle gas flow rate and relatively high overall conversion of approximately eighty-five percent while maintaining a relatively low power requirement for recycle compressor 240. In the exemplary embodiment, the low per pass conversion and high recycle rate enables a lower height of reactor 216, which provides a more uniform top to bottom gas composition, a more uniform flow distribution with less channeling, a more uniform catalyst distribution, and a more uniform temperature profile across reactor 216.

More uniform FT reactor 216 conditions facilitate reducing FT product variation from the desired kero and diesel range (C10 to C20). Wax production is minimized allowing a small base lube oil hydrocracker (pipe reactor) to be added to a return wax stream 266 from an FT catalyst recovery system 268, where waxy products that accumulate in the catalyst pores are separated. Return wax stream 266, including un-reacted hydrogen and light hydrocarbons, is channeled to FT distillate stripper 232 and atmospheric distillation column 252. FT distillate stripper 232 separates out the light components (H₂, C1-C4), which are combined with FT tail gas stream 242 and routed to gas separation system 246. The heavier components are fractionated to finished products stream 254 (including lube oil base stock) in atmospheric distillation column 252.

Exemplary embodiments of carbon to liquids systems and methods of minimizing liquid product variation from the Fischer-Tropsch reactor are described above in detail. The carbon to liquids system components illustrated are not limited to the specific embodiments described herein, but rather, components of each system may be utilized independently and separately from other components described herein. For example, the carbon to liquids system components described above may also be used in combination with different carbon to liquids system components.

The above-described carbon to liquids systems and methods are cost-effective and highly reliable. The method permits a reduced of variation of FT liquids product boiling point and carbon number and facilitates minimizing wax production. The method also provides improved diesel selectivity over naphtha due to higher operating pressure, a relatively high catalyst activity due to low water vapor concentration and substantially eliminates a need for external knockout and water separation drums. The carbon to liquids system provides a reduced FT reactor height and catalyst volume, a reduced FT liquids product boiling point (carbon number) variation, a significantly reduced wax production, which permits wax removal to be integrated into the catalyst regeneration system thereby eliminating a separate hydrocracker upgrading unit. Accordingly, the systems and methods described herein facilitate the operation of carbon to liquids systems in a cost-effective and reliable manner.

While the invention has been described in terms of various specific embodiments, those skilled in the art will recognize that the invention can be practiced with modification within the spirit and scope of the claims.

What is claimed is:

1. A method of operating a carbon to liquids system comprising:
receiving a flow of syngas;
shifting the syngas to increase an H₂/CO ratio of the syngas;
mixing hydrogen with the shifted syngas to increase the H₂/CO ratio;
reacting the hydrogen/shifted syngas mixture with a catalyst in a vessel at a pressure of approximately 600 psia such that approximately 40% of the hydrogen/shifted syngas mixture is converted; and
recycling an unreacted hydrogen/shifted syngas mixture to the vessel.

2. A method in accordance with claim 1 wherein receiving a flow of syngas comprises receiving a flow of syngas from a coal gasification process.

3. A method in accordance with claim 1 wherein receiving a flow of syngas comprises receiving a flow of syngas having an H₂/CO ratio of approximately 1.6.

4. A method in accordance with claim 1 wherein shifting the syngas comprises shifting the syngas to an H₂/CO ratio of approximately 1.85.

5. A method in accordance with claim 1 wherein mixing hydrogen with the shifted syngas to increase the H₂/CO ratio to approximately 2.1.

6. A method in accordance with claim 1 wherein reacting the hydrogen/shifted syngas mixture with a catalyst comprises reacting the hydrogen/shifted syngas mixture with a catalyst in a Fischer Tropsch synthesis reactor.

7. A method in accordance with claim 1 wherein reacting the hydrogen/shifted syngas mixture with a catalyst comprises reacting the hydrogen/shifted syngas mixture with a catalyst in a slurry bubble column reactor type Fischer-Tropsch synthesis reactor.

8. A method in accordance with claim 1 wherein mixing hydrogen comprises mixing hydrogen from a flow of recycled tail gas.

9. A method in accordance with claim 1 wherein reacting the hydrogen/shifted syngas mixture with a catalyst comprises reacting the hydrogen/shifted syngas mixture with a catalyst such that a water partial pressure in the vessel is low enough to substantially reduce oxidizing and deactivating the catalyst.

10. A method in accordance with claim 1 wherein recycling an un-reacted hydrogen/shifted syngas mixture to the vessel comprises recycling an un-reacted hydrogen/shifted syngas mixture through a condenser having a relatively low pressure drop permitting a relatively high recycle gas flow rate.

11. A method in accordance with claim 1 further comprising recycling naphta to facilitate catalyst recovery.

12. A carbon to liquids system comprising:
a source of syngas;
a vessel configured to shift the syngas to increase an H₂/CO ratio of the syngas, said vessel coupled in flow communication downstream of said source of syngas;
a source of gas comprising hydrogen coupled in flow communication with the shifted syngas, said source of gas configured to be mixed with said shifted syngas to increase the H₂/CO ratio of the shifted syngas;
a vessel comprising an inlet and an outlet, said inlet configured to receive the gas and shifted syngas mixture, said vessel comprising a catalyst configured to facilitate a Fischer-Tropsch synthesis reaction at a pressure of approximately 600 psia such that approximately 40% of the hydrogen/shifted syngas mixture is converted; and
a recycle path communicatively coupled between said outlet and inlet configured to channel an un-reacted hydrogen/shifted syngas mixture to the vessel inlet.

13. A system in accordance with claim 12 further comprising a gasifier configured to generate a flow of syngas from a carbonaceous fuel comprising coal.

14. A system in accordance with claim 12 wherein said shifted syngas comprises an H₂/CO ratio of between approximately 1.7 to approximately 1.95.

15. A system in accordance with claim 12 wherein said shifted syngas comprises an H₂/CO ratio of approximately 2.1.

16. A system for generating liquid hydrocarbons from gaseous reactants comprising:
a source of syngas comprising hydrogen and carbon monoxide in a ratio of between approximately 1.4 and approximately 1.8;
a shift reactor configured to shift the syngas to increase an H₂/CO ratio of the syngas, said vessel coupled in flow communication downstream of said source of syngas;
a source of gas comprising hydrogen coupled in flow communication with the shifted syngas, said source of gas configured to be mixed with said shifted syngas to increase the H₂/CO ratio of the shifted syngas to between approximately 1.9 to approximately 2.3;
a vessel comprising an inlet and an outlet, said inlet configured to receive the gas and shifted syngas mixture, said vessel comprising a catalyst configured to facilitate a Fischer-Tropsch synthesis reaction at a pressure of approximately 600 psia such that approximately 40% of the hydrogen/shifted syngas mixture is converted; and
a recycle path communicatively coupled between said outlet and inlet, said recycle path configured to channel an un-reacted hydrogen/shifted syngas mixture to the vessel inlet.

17. A system in accordance with claim 16 wherein said shifted syngas comprises an H₂/CO ratio of between approximately 1.7 to approximately 1.95.

18. A system in accordance with claim 16 wherein said hydrogen/shifted syngas mixture comprises an H₂/CO ratio of approximately 2.1.

19. A system in accordance with claim 16 wherein said vessel comprises a slurry bubble column reactor type Fischer-Tropsch synthesis reactor.

20. A system in accordance with claim 16 wherein said vessel is further configured to maintain a water partial pressure in the vessel below a predetermined threshold to facilitate reducing oxidization and deactivation of the catalyst.

21. A system in accordance with claim 16 wherein said recycle path further comprises a condenser configured to operate with a low pressure drop.

22. A method in accordance with claim 21 wherein said condenser facilitates recycling of supercritical naphta.

23. A method in accordance with claim 21 wherein said condenser facilitates catalyst recovery.
